(19) United States
(12) Patent Application Publication
(10) Pub. No.: US 2008/0317194 A1
(43) Pub. Date: Dec. 25, 2008

(54) Pet Imaging Using Anatomic List Mode Mask

(75) Inventors: Daniel Gagnon, Twinsburg, OH (US); Wenli Wang, Aurora, OH (US); Zhiqiang Hu, Twinsburg, OH (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/093,152

(22) PCT Filed: Oct. 17, 2006
(86) PCT No.: PCT/IB06/53824

§ 371 Date: May 9, 2008

Related U.S. Application Data
(60) Provisional application No. 60/597,105, filed on Nov. 10, 2005.

(51) Int. Cl. A61B 6/00

(52) U.S. Cl. 378/4

(57) ABSTRACT

A method and apparatus are provided for reconstructing list mode data acquired during a positron emission tomography scan of an object, the data including information indicative of a plurality of detected positron annihilation events. Detected events occurring in a region of interest are identified; the identified events are reconstructed using an iterative reconstruction technique which includes a ray tracing operation to generate volumetric data indicative of the region of interest, wherein the ray tracing operation traces only image matrix elements located in the region of interest; and a human readable image indicative of the volumetric data is generated. In another aspect an image mask and a projection mask are defined correlating to the region of interest; image matrix elements located in the region of interest are determined by applying the image mask; and detected events occurring in a region of interest are identified by applying the projection mask.

The present invention relates to the field of positron imaging, and more particularly to the reconstruction of list mode data acquired in positron emission tomography (PET).

Positron emission tomography (PET) is a branch of nuclear medicine in which a positron-emitting radiopharmaceutical such as ¹⁸F-fluorodeoxyglucose (FDG) is introduced into the body of a patient. As the radiopharmaceutical decays, positrons are generated. More specifically, each of a plurality of positrons reacts with an electron in what is known as a positron annihilation event, thereby generating a coincident pair of 511 keV gamma rays which travel in opposite directions along a line of response (LOR). A gamma ray pair detected within a coincidence time is ordinarily recorded by the PET scanner as an annihilation event. In time of flight (TOF) imaging, the time within the coincidence interval at which each gamma ray in the coincident pair is detected is further measured. The time of flight information provides an indication of the location of the detected event along the LOR.

Annihilation event data from the scan is used to reconstruct volumetric data indicative of the distribution of the radionuclide in the object. Reconstruction is typically performed using statistical (iterative) or analytical reconstruction algorithms. Iterative methods can provide a superior reconstruction relative to analytical reconstruction methods. However, they are as a rule more complex, computationally more expensive, and relatively more time consuming. Iterative reconstruction techniques include the maximum likelihood expectation maximization (ML-EM), ordered subsets expectation maximization (OS-EM), resealed block iterative expectation maximization (RBI-EM), and row action maximization likelihood (RAMLA) techniques. See Shepp and Vardi, Maximum Likelihood Reconstruction for Emission Tomography, IEEE Trans. Med. Imaging vol. MI-2, pp 113-122 (1982); Hudson and Larkin, Accelerated Image Reconstruction Using Ordered Subsets of projection Data, IEEE Trans. Med. Imaging vol. 13, no. 4, pp 601-609 (1994); Byrne, Accelerating the EMML Algorithm and Related Iterative Algorithms by Rescaled Block-Iterative Methods, IEEE Trans. Image Processing, vol. 7, no. 1 pp. 100-109 (1998); Brown and DePierro, A Row-Action Alternative to the EM Algorithm for Maximizing Likelihoods in Emission Tomography, IEEE Trans. Med. Imaging vol. 15, no. 5, pp 687-699 (1996).

Reconstruction time can be a key factor in the performance of PET imaging systems. This is especially true when iterative reconstruction techniques are used. Iterative reconstruction can be characterized as involving three basic steps: first, starting with an initial object estimate an object estimate is forward projected into the projection domain; second, the resulting projections are compared against the measured projections to form corrections in the projection domain; and third, the corrections are then backprojected into the image domain and used to update the object estimate. The three basic steps thus described are repeated through additional iterations until the estimate converges to a solution or the iteration process is otherwise terminated.

Generally the total reconstruction time on list mode data is proportional to the total number of events to be processed. Thus one technique for reducing reconstruction time is to reduce the amount of annihilation event data utilized. Reported annihilation events which occur outside of the boundaries of an object or region of interest under examination are generally error events, commonly scatter and random events, and may be disregarded during reconstruction. Also, reconstruction efficiencies may also be accomplished by limiting updating of image domain elements (for example voxels, blobs or other basis functions) to elements within the boundaries of an object or region of interest under examination, thereby reducing reconstruction times.

However, if list mode event data or image domain elements are identified for exclusion through techniques executed during reconstruction, reconstruction iteration steps and resources must still be expended prior to identification. Accordingly, it is desirable that list mode event data and/or image elements outside of object or region-of-interest boundaries be identified for exclusion prior to reconstruction, thereby improving overall reconstruction times.

Aspects of the present invention address these matters, and others.

A method and apparatus are provided for reconstructing list mode data acquired during a positron emission tomography scan of an object, the data including information indicative of a plurality of detected positron annihilation events. Detected events occurring in a region of interest are identified; the identified events are reconstructed using an iterative reconstruction technique which includes a ray tracing operation to generate volumetric data indicative of the region of interest, wherein the ray tracing operation traces only image matrix elements located in the region of interest; and a human readable image indicative of the volumetric data is generated.

In another aspect an image mask and a projection mask are defined correlating to the region of interest; image matrix elements located in the region of interest are determined by applying the image mask; and detected events occurring in a region of interest are identified by applying the projection mask. In another aspect image matrix elements outside of image mask boundaries are assigned out-of-boundary values.

In another aspect non-PET imaging modality scan data indicative of the object is acquired, and the image mask is defined by mapping the non-PET imaging modality scan data to PET image element dimensions and segmenting the mapped data. In another aspect the projection mask is defined by mapping the non-PET imaging modality scan data to PET image element dimensions; forward-projecting the mapped non-PET imaging modality scan data into projection space; and thresholding the forward-projected data.

In another aspect both projection mask and image mask are larger than the region of interest.

In another aspect the plurality of positron annihilation events comprises list mode LOR data, the identified events reconstructed by determining if an LOR is located within the projection mask, and if the LOR is located within the projection mask then using the LOR to trace image elements not having an out-of-boundary value.

In another aspect the plurality of positron annihilation events comprises list mode LOR data including TOF information, the identified events reconstructed by using the TOF information to determine an occurrence probability that an annihilation event represented by an LOR is located within the projection mask. If the occurrence probability indicates that the annihilation event represented by the LOR is located within the projection mask, then the LOR is used to trace image elements not having an out-of-boundary value.

Those skilled in the art will appreciate still other aspects of the present invention upon reading and understanding the appended description.

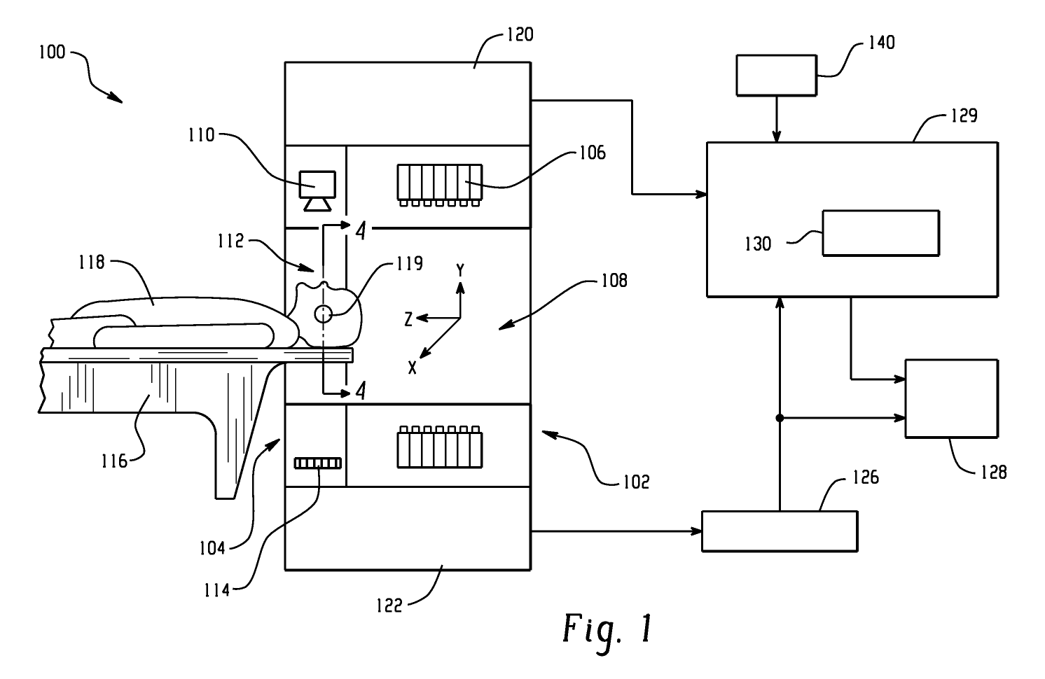

FIG. 1 depicts a combined PET/CT system.

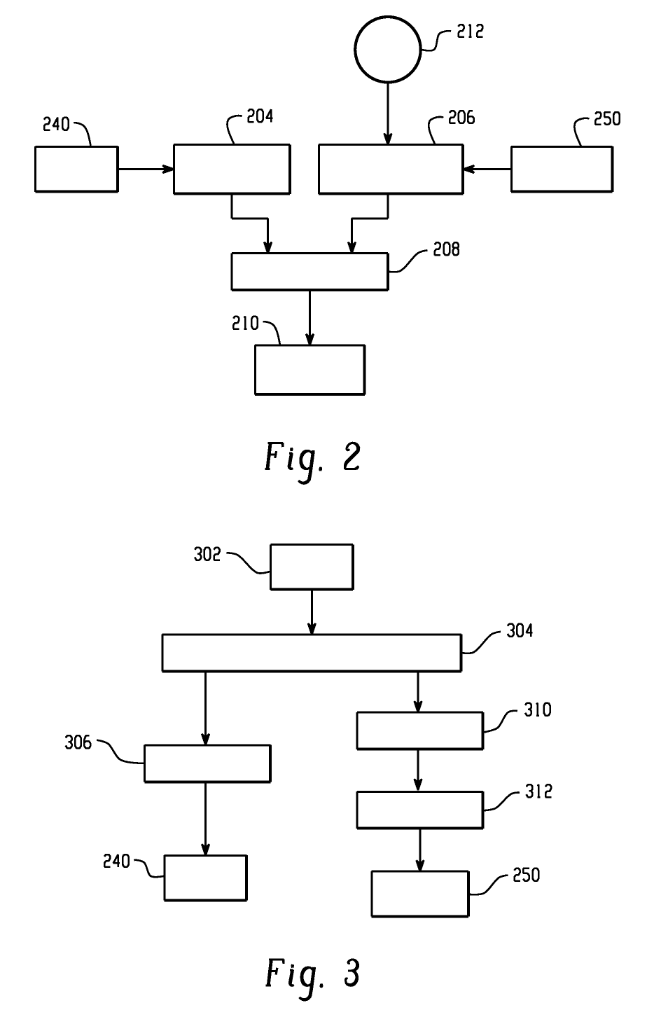

FIG. 2 is a flowchart of a PET image reconstruction method.

FIG. 3 is a flowchart of anatomic mask determination.

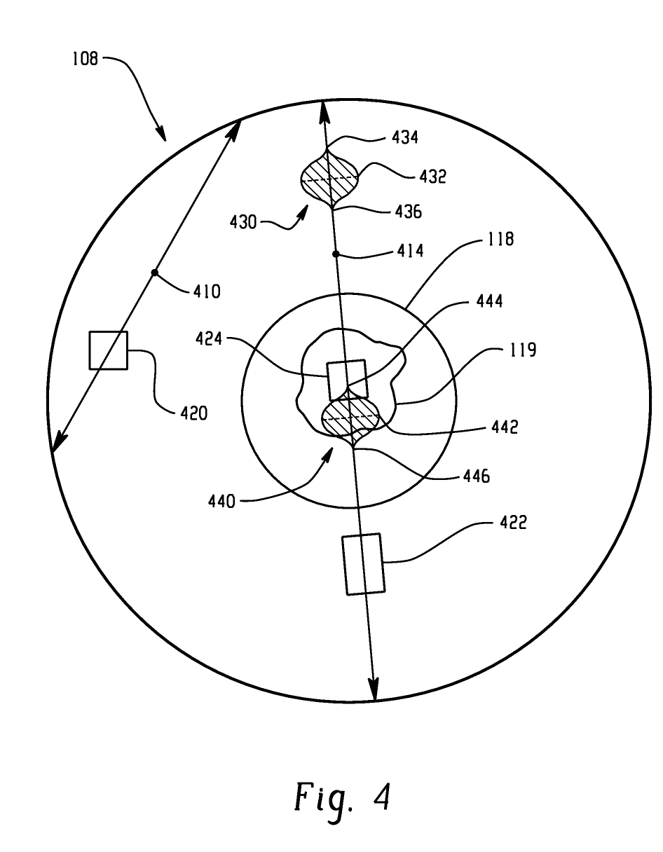

FIG. 4 is partial cross-sectional view illustration of the combined PET/CT system of FIG. 1 taken along the lines indicated in FIG. 1, and incorporating additional illustrative elements.

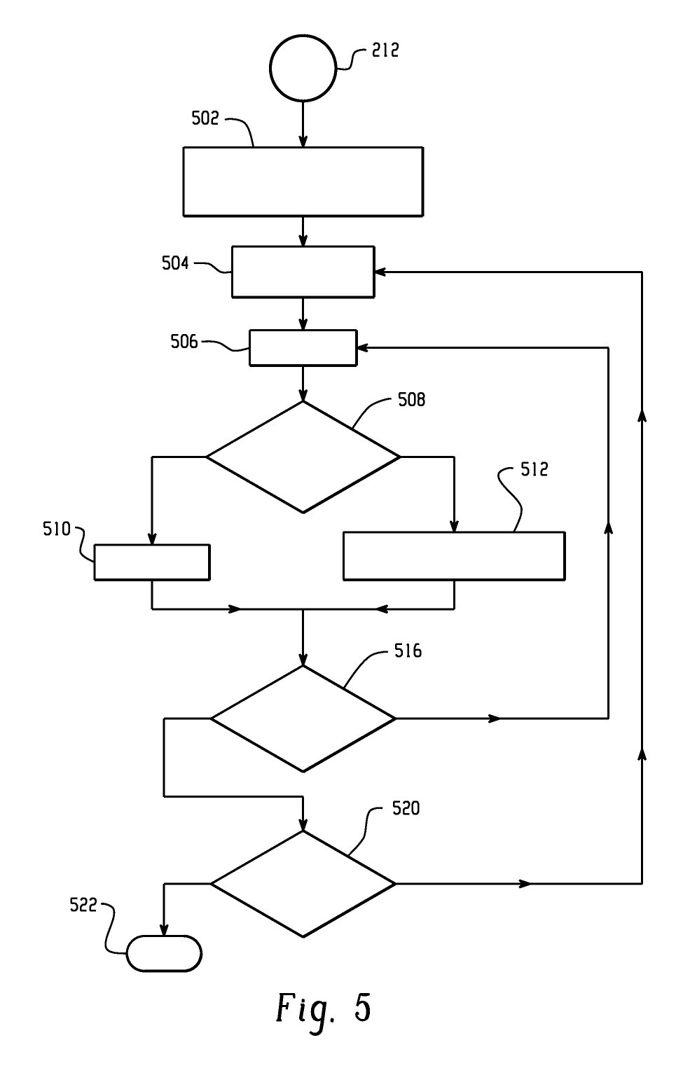

FIG. 5 is a flowchart of an application of an image mask and a projection mask to list mode event data.

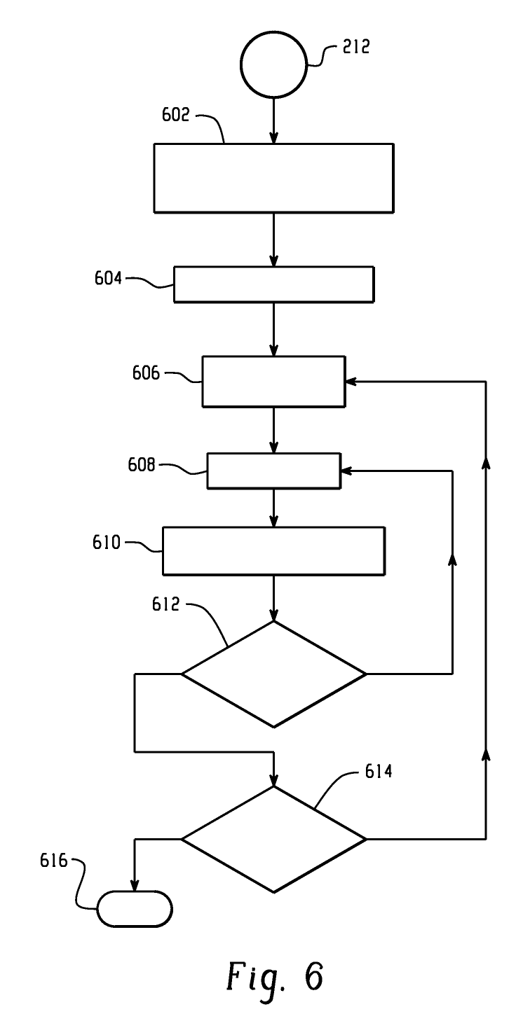

FIG. 6 is a flowchart of another application of an image mask and a projection mask to list mode event data.

With reference to FIG. 1, a combined PET/CT system 100 includes a PET gantry portion 102 and a CT gantry portion 104. The PET gantry portion 102 includes one or more axial rings of radiation sensitive detectors 106 which surround an examination region 108. The detectors 106 detect gamma radiation characteristic of positron annihilation events occurring within a PET examination region 108.

The CT portion 104 includes a radiation source 110 such as an x-ray tube which rotates about a CT examination region 112. Radiation sensitive detectors 114 detect radiation emitted by the x-ray source which has traversed the examination region 112.

The PET gantry portion 102 and CT gantry portion 104 are preferably located in proximity with their respective examination regions 108, 112 disposed along a common longitudinal or z-axis. An object support 116 supports an object to be imaged 118 such as human patient. The object support 116 is preferably longitudinally movable in coordination with operation of the PET/CT system 100 so that the object 118 can be scanned at a plurality of longitudinal locations by both the PET and CT gantry portions 102, 104.

A CT data acquisition system 122 processes the signals from the CT detectors 114 to generate data indicative of the radiation attenuation along a plurality of lines or rays through the examination region 112. A CT reconstructor 126 reconstructs the data using suitable reconstruction algorithms to generate volumetric image data indicative of the radiation attenuation of the object 118.

PET data acquisition system 120 provides projection data which includes a list of annihilation events detected by the detectors 106. More particularly, the projection data provides information on the LOR for each event, such as a transverse and longitudinal position of the LOR, its transverse and azimuthal angles, and TOF information. Alternately, the data may be rebinned into one or more sinogram or projection bins.

A PET reconstructor 129 includes at least one computer or computer processor 130. Generally speaking, the use of additional or more powerful processors will improve reconstruction speed. The reconstructor 129 uses an iterative technique to generate volumetric image data indicative of the distribution of the radionuclide in the object 118. Suitable techniques include ML-EM, OS-EM, RBI-EM, and RAMLA, although other techniques may be implemented. One exemplary iterative reconstruction model is an ML-EM algorithm expressed as follows:

$${x}_{i}^{n + 1} = \frac{{x}_{i}^{n}}{{\sum }_{j = 1}^{J} {a}_{ij}} {\sum }_{j = 1}^{J} {α}_{ij} \frac{{p}_{j}}{{\sum }_{k} {a}_{kj} {x}_{k}^{n}} \qquad Equation 1$$

Where xⁿᵢ is the image estimate of the 1^{th} volumetric element, such as a voxel or blob, for the n^{th} iteration, pⱼ is the j^{th} projection data, aᵢⱼ the system matrix element representing the possibility of detecting a photon pair in the j^{th} projection given an emission from the i^{th} volumetric element.

In addition, the PET reconstructor 129 preferably uses information from the CT reconstructor 126 to apply attenuation and other desired corrections to the PET data. Computer readable instructions which cause the processor or processors 130 to carry out the reconstruction are preferably carried on one or more computer readable media 140 such as computer disks, volatile or non-volatile memory, or the like, and may also be transmitted by way of a suitable communications network such as the internet to storage media 140 accessible to the processor(s) 130.

A workstation computer serves as an operator console 128. The console 128 includes a human readable output device such as a monitor or display and input devices such as a keyboard and mouse. Software resident on the console 128 allows the operator to view and otherwise manipulate the volumetric image data generated by the PET and CT reconstructors 129,126. Software resident on the console 128 also allows the operator to control the operation of the system 100 by establishing desired scan protocols, initiating and terminating scans, and otherwise interacting with the scanner.

FIG. 2 presents a generalized reconstruction technique performed by the reconstructor 129. At 204 an image mask 240 is used to set elements in an image matrix falling outside the object 118 boundary or a region of interest 119 within the object to an out-of-boundary value. The region-of-interest 119 may be a predetermined area within the object 118, such as an anatomic region defined by one or more specified internal organs or organ portions. In the exemplary embodiments illustrated and discussed below the out-of-boundary value is zero; however, it is to be understood that other values or threshold values may be used to indicate an out-of-boundary element.

At 206 a projection mask 250 is applied to PET list mode event data 212 to exclude events outside of the object under examination 118 or a desired region of interest 119 within the object. At 208, the PET events 212 less the events excluded through application of the projection mask 250 is reconstructed using the MLEM or other suitable iterative reconstruction technique to generate volumetric image data, wherein ray tracing operations in forward and/or backprojection steps of the reconstruction process do not update image matrix elements initialized to zero through application of the image mask 240. The present technique is also applicable to histogram event data. At 210 a final image estimate is made available.

Thus the identification and exclusion of list mode events outside of scanned object 118 or region-of-interest 119 boundaries through application of the projection mask 250 correspondingly reduces total reconstruction time in step 208, relative to alternative reconstruction techniques that generate image estimates from all list mode events 212 inclusive of events occurring outside of the scanned object 118 or region-of-interest 119. And identification of image elements outside of scanned object 118 or region-of-interest 119 boundaries during the image matrix initialization at 204 through application of the image mask 240 enables exclusion of the identified image elements from updating during reconstruction in step 208, providing improved reconstruction efficiencies relative to alternative reconstruction techniques that update all image elements inclusive of image elements occurring outside of the scanned object 118 or region-of-interest 119.

FIG. 3 illustrates a method for determining the anatomic image mask 240 and the projection mask 250. At 302 CT volumetric image data is provided by the CT reconstructor 126 as described generally above. At 304 the CT volumetric image data is segmented to identify the boundaries of the object 118 or of a region of interest 119 in the object. The segmented data is also registered with the PET system and remapped as needed to match PET image element dimensions. As indicated at step 306, the segmentation may be performed by thresholding, although special care should be taken to identify voxels near to the object boundary having values similar to air (e.g., the lungs). Other suitable segmentation techniques may also be used. The image mask 240 thus describes the extent of the object 118 or a region of interest therein.

At 310, the segmented data generated at 304 is forward projected into the projection domain to generate a three-dimensional attenuation sinogram. Thresholding at 312 then produces a projection mask 250 having the same dimensions as the emission sinogram for application to the event data 212 in projection domain. To avoid artifacts resulting from boundary conditions, it is preferred to define the projection mask 250 larger than the actual object or region-of-interest boundaries observed. The projection domain is represented as list mode, although the technique may be adapted to sinogram or projection data, or in any other suitable manner.

Referring now to FIGS. 2 and 4, a LOR 410 occurs outside of the boundaries of a region-of-interest 119. Application of the projection mask 250 at 206 as discussed above identifies LOR 410 as occurring outside of the region-of-interest 119 boundaries. Accordingly, the LOR 410 will be excluded from reconstruction at step 208.

Another LOR 414 intersects the region-of-interest 119, and passes through both voxel 422 located outside of the region-of-interest 119 boundaries and voxel 424 located inside the region-of-interest 119 boundaries. Application of the projection mask 250 at 206 does not identify LOR 414 for exclusion as occurring outside of object 118 and region-of-interest 119 boundaries, and LOR 414 is included in reconstruction at step 208.

In another aspect, a determination that an associated annihilation event actually occurs within the projection mask 250 may be utilized to determine exclusion of the LOR 414. In one technique TOF information associated with LOR 414 may be used to determine a probability that an annihilation event represented by LOR 414 occurs at a point along LOR 414 within the region-of-interest 119. Accordingly, in a first example TOF information indicates that LOR 414 represents an annihilation event occurring within LOR segment 430 along LOR 414, with a highest probability indicating occurrence at midpoint 432 and lowest probabilities of occurrence at endpoints 434 and 436. As the LOR segment 430 does not occur within the region of interest 119, LOR 414 is excluded during reconstruction at step 208.

In a second example TOF information indicates that LOR 414 represents an annihilation event occurring within LOR segment 440 along LOR 414, with a highest probability indicating occurrence at midpoint 442 and lowest probabilities of occurrence at endpoints 444 and 446. As the LOR segment 440 does occur at least in part within the region of interest 119, LOR 414 is not excluded but is traced during reconstruction at step 208.

In another aspect the lowest probabilities of occurrence at endpoints 434, 436, 444 and 446 may be zero, or they may be a boundary probability value selected responsive to one or more parameters. On example of a parameter is a specified image resolution requirement; other parameters will be apparent to one skilled in the art.

In another aspect during initialization of the image matrix for the iterative reconstruction, application of the image mask 240 identifies voxel 422 as falling outside the image mask 240 and sets its value to zero. During reconstruction step 208 ray tracing performed as part of forward and backprojection operations the identified voxels remain at zero or are otherwise not updated. It is to be understood that this technique is not limited to voxel applications, and is appropriate for other basis functions, such as blobs.

Accordingly, since the voxel 422 value is zero, where LOR 414 is not excluded by application of the projection mask 250 voxel 422 is nevertheless not updated by tracing LOR 414 at reconstruction step 208. In contrast, voxel 424 occurs inside of the region-of-interest 119 boundaries and accordingly its value is not set to zero by application of the image mask 240 at 204: voxel 424 is therefore updated by tracing LOR 414 at reconstruction step 208. As will be appreciated, reducing the number of image elements updated in any given reconstruction iteration by disregarding events outside of the region-of-interest 119 boundaries, and not updating image space volumetric elements located outside image space volumetric elements responsive to events occurring within the region-of-interest 119 boundaries reduces the time required to complete forward and backprojection operations, thereby reducing reconstruction time and providing efficiency advantages.

In one aspect the relative ordering of the image mask 240 application step 204 and projection mask 250 application step 206 as described above is not required, and the order of these steps may be reversed. In another aspect the image mask 240 may be applied to all image domain elements prior to ray tracing iterations, providing prior filtering of all image domain elements and thereby setting voxel 420 value to zero. Alternative techniques may defer application of the image mask 240 until selection of a voxel for updating through tracing of one or more LOR's after application of the projection mask 250: thus if no LOR is determined to pass through voxel 420, then voxel 420 is not compared to the image mask 240 and its value is not set to zero. Moreover, in another aspect either or both masks 240, 250 may be applied during the reconstruction step 208.

FIG. 5 illustrates a reconstruction technique as applied to a plurality of list mode LOR event data 212. At 502 an image matrix is initialized, wherein volumetric image elements falling outside of the image mask 240 have their values set to zero. At 504 a reconstruction iteration is initiated. A LOR is then selected at 506 and compared to the projection mask 250 at 508. If the LOR is not within the projection mask 250, then at 510 the LOR is excluded from further processing. In another aspect if TOF information indicates that the LOR represents an annihilation event not occurring within the projection mask 250, then at 510 the LOR is excluded from further processing. Alternatively, if the LOR is within the projection mask 250 as determined at 508, and/or if TOF information indicates a probability greater than a boundary probability value that the LOR represents an annihilation event occurring within the projection mask 250, then the LOR is processed as part of the reconstruction. As indicated at 512, only those image elements having a value greater than zero are updated during the ray tracing process.

As reflected by step 516, if all of the LORs have not been selected, processing is returned to step 506 and the next LOR selected. As reflected by step 520, each LOR is again selected for steps 504, 506, 508, 510 or 512 and 516 through successive iterations, until the object estimate converges, a desired number of iterations have been performed, or reconstruction terminates at 522. The most recent object estimate becomes the final object estimate at 520. The final object estimate is stored in suitable memory and made available to the operator console computer 128 for further display, processing, and/or analysis. The reconstructed image data may also be made available to other computers associated with the scanner or otherwise having access to a common network such as a picture archiving and communication (PACS) system, hospital information system/radiology information system (HIS/RIS) system, the internet, or the like.

FIG. 6 illustrates another reconstruction technique as applied to a plurality of list mode events 212. At 602 an image matrix is initialized, wherein each volumetric image element in the matrix which falls outside the image mask 240 has its value set to zero. Next the projection mask 250 is applied at 604 to each event to identify those events occurring outside of the object 118 or region-of-interest 119 boundaries. Thus the events 212 are filtered through the projection mask 250 and only those events occurring within the object 118 or region-of-interest 119 boundaries are used in the reconstruction.

A reconstruction iteration is initiated at 606. Each event is selected at 608 for tracing. And only image elements having a value greater than zero are updated by ray tracing of the selected event at 610.

As reflected by step 612, each event is selected at 608 for updating of non-zero image elements at 610 until all events have been selected. As reflected by step 614, each event is again selected for additional iterations until the object estimate converges, a desired number of iterations have been performed, or reconstruction terminates at 616. The most recent object estimate becomes the final object estimate at 614, available as discussed above with respect to step 520.

Although CT imaging has been discussed thus far in providing anatomical object information for determination of the image mask 240 and projection mask 250, it should be appreciated that other non-PET imaging modality techniques may be utilized to acquire anatomic object information. For example, the CT portion of the scanner 100 may be omitted and replaced with another imaging device such as a magnetic resonance (MR) scanner. Alternately, attenuation or anatomical information may be provided by a transmission source associated with the PET gantry portion 102, such as for example magnetic resonance (MR) resolution techniques.

An embodiment of the invention described above is tangibly embodied in a computer program stored in suitable memory storage device 140 and made available to the system 100 and reconstructor 129. Exemplary machine-readable memory storage mediums include, but are not limited to, fixed hard drives, optical discs, magnetic tapes, semiconductor memories, such as read-only memories (ROMs), programmable (PROMs), etc. The memory 140 containing the computer readable code is utilized by executing the code directly from the memory 140, or by copying the code from one memory storage device to another memory storage device, or by transmitting the code on a network for remote execution. The memory 140 may comprise one or more of a fixed and/or removable data storage device such as a floppy disk or a CD-ROM, or it may consist of some other type of data storage or data communications device. The computer program may be loaded into the memory of a computer to configure a processor for execution of the techniques described above. The computer program comprises instructions which, when read and executed by a processor causes the processor to perform the steps necessary to execute the steps or elements of the present invention.

The invention has been described with reference to the preferred embodiments. Of course, modifications and alterations will occur to others upon reading and understanding the preceding description. It is intended that the invention be construed as including all such modifications and alterations insofar as they come within the scope of the appended claims.

1. A method for reconstructing list mode data acquired during a positron emission tomography scan of an object, the data including information indicative of a plurality of detected positron annihilation events, the method comprising:
identifying detected list mode events occurring in a region of interest;
reconstructing the identified list mode events using an iterative reconstruction technique which includes a ray tracing operation to generate tomographic data indicative of the region of interest, wherein the ray tracing operation traces only image matrix elements located in the region of interest; and
generating an image indicative of the tomographic data.

2. The method of claim 1, further comprising the step of defining a projection mask which correlates to the region of interest;
wherein the step of identifying detected list mode events occurring in a region of interest comprises applying the projection mask to the plurality of detected positron annihilation events.

3. The method of claim 2, further comprising the steps of:
defining an image mask which correlates to the region of interest; and
using the image mask to identify the image matrix elements located in the region of interest by assigning an out-of-boundary value to image matrix elements located outside the region of interest.

4. The method of claim 3, wherein the step of defining the image mask comprises the steps of:
obtaining non-PET imaging modality scan data indicative of the object;
mapping the non-PET imaging modality scan data to PET image element dimensions; and
segmenting the mapped non-PET imaging modality scan image data.

5. The method of claim 2, wherein the step of defining the projection mask comprises the steps of:
obtaining non-PET imaging modality scan data indicative of the object;
forward-projecting the non-PET imaging modality scan data into projection space; and
thresholding the forward-projected data.

6. The method of claim 3, wherein both the projection mask and the image mask are larger than the region of interest.

7. The method of claim 3, wherein the plurality of positron annihilation events comprises a plurality of list mode LORs, and the step of reconstructing further comprises the steps of:
determining if an LOR is located within the projection mask; and
if the LOR is located within the projection mask then using the LOR to trace image elements not having the out-of-boundary value.

8. The method of claim 3, wherein the plurality of positron annihilation events comprises a plurality of list mode LORs including TOF information, and the step of reconstructing further comprises the steps of:
using the TOF information to determine an occurrence probability that an annihilation event represented by an LOR is located within the projection mask; and
if the occurrence probability indicates that the annihilation event represented by the LOR is located within the projection mask, then using the LOR to trace image elements not having the out-of-boundary value.

9. The method of claim 2, wherein the step of reconstructing the identified list mode events comprises the steps of:
using annihilation event TOF information to determine an occurrence probability that an event represented by an LOR is located within the projection mask; and
if the occurrence probability indicates that the annihilation event represented by the LOR is located within the projection mask, then using the LOR to trace image elements located in the region of interest.

10. An apparatus for reconstructing list mode data acquired during a positron emission tomography scan of an object, the data including information indicative of a plurality of detected positron annihilation events, comprising:
a reconstructor means configured to identify detected list mode events occurring in a region of interest;
the reconstructor means (further configured to reconstruct the identified list mode events using an iterative reconstruction technique which includes a ray tracing operation to generate tomographic data indicative of the region of interest, wherein the ray tracing operation traces only image matrix elements located in the region of interest; and
a display means for generating an image indicative of the tomographic data.

11. The apparatus of claim 10, wherein the reconstructor means is further configured to:
define a projection mask which correlates to the region of interest; and
identify the detected list mode events occurring in a region of interest by applying the projection mask to the plurality of detected positron annihilation events.

12. The apparatus of claim 11 wherein the reconstructor means is further configured to:
define an image mask which correlates to the region of interest; and
use the image mask to identify image matrix elements located in the region of interest by assigning an out-of-boundary value to image matrix elements located outside the region of interest.

13. The apparatus of claim 12, further comprising a non-PET imaging modality data acquisition system, wherein the reconstructor means is further configured to define the image mask by:
mapping non-PET imaging modality scan data received from the non-PET imaging modality data acquisition system indicative of the object to PET image element dimensions; and
segmenting the mapped non-PET imaging modality scan data.

14. The apparatus of claim 11, further comprising a non-PET imaging modality data acquisition system 122, wherein the reconstructor means is further configured to define the projection mask by:
forward-projecting non-PET imaging modality scan data received from the non-PET imaging modality data acquisition system (44% indicative of the object into projection space; and
thresholding the forward-projected data.

15. The apparatus of claim 12, wherein both the projection mask and the image mask are larger than the region of interest.

16. The apparatus of claim 12, wherein the reconstructor means is configured to reconstruct list mode LOR data by:
determining if an LOR is located within the projection mask; and
if the LOR is located within the projection mask then using the LOR to trace image elements not having the out-of-boundary value.

17. The apparatus of claim 12, wherein reconstructor means is configured to reconstruct list mode LOR data by:
using the TOF information to determine an occurrence probability that an annihilation event represented by an LOR is located within the projection mask; and
if the occurrence probability indicates that the annihilation event represented by the LOR is located within the projection mask, then using the LOR to trace image elements not having the out-of-boundary value.

18. The apparatus of claim 11 wherein the reconstructor means is further configured to reconstruct the identified list mode events by:
using annihilation event TOF information to determine an occurrence probability that an event represented by an LOR is located within the projection mask; and
if the occurrence probability indicates that the annihilation event represented by the LOR is located within the projection mask, then using the LOR to trace image elements located in the region of interest.

19. An article of manufacture comprising a computer usable medium having a computer readable program embodied in said medium, wherein the computer readable program, when executed on a computer, causes the computer to reconstruct list mode data acquired during a positron emission tomography scan of an object, the list mode data including information indicative of a plurality of detected positron annihilation events, by:
identifying detected list mode events occurring in a region of interest;
reconstructing the identified list mode events using an iterative reconstruction technique which includes a ray tracing operation to generate tomographic data indicative of the region of interest, wherein the ray tracing operation traces only image matrix elements located in the region of interest; and
generating an image indicative of the tomographic data.

20. The article of manufacture (44% of claim 19, wherein the computer readable program, when executed on the computer, further causes the computer to define a projection mask which correlates to the region of interest;
wherein the computer further identifies the detected list mode events occurring in a region of interest by applying the projection mask to the plurality of detected positron annihilation events.

21. The article of manufacture (144 of claim 20, wherein the computer readable program, when executed on the computer, further causes the computer to:
define an image mask which correlates to the region of interest; and
use the image mask to identify image matrix elements located in the region of interest by assigning an out-of-boundary value to image matrix elements located outside the region of interest.

22. The article of manufacture of claim 21, wherein the computer readable program, when executed on the computer, further causes the computer to define the image mask by:
mapping non-PET imaging modality scan data received from a non-PET imaging modality data acquisition system indicative of the object to PET image element dimensions; and
segmenting the mapped non-PET imaging modality scan data.

23. The article of manufacture of claim 20, wherein the computer readable program, when executed on the computer, further causes the computer to define the projection mask by:
forward-projecting non-PET imaging modality scan data received from a non-PET imaging modality data acquisition system indicative of the object into projection space; and
thresholding the forward-projected data.

24. The article of manufacture (of claim 21, wherein the computer readable program, when executed on the computer, further causes the computer to define both the projection mask and the image mask larger than the region of interest.

25. The article of manufacture % of claim 21, wherein the computer readable program, when executed on the computer, further causes the computer to reconstruct LOR data by:
determining if an LOR is located within the projection mask; and
if the LOR is located within the projection mask then using the LOR to trace image elements not having the out-of-boundary value.

26. The article of manufacture (of claim 21, wherein the computer readable program, when executed on the computer, further causes the computer to reconstruct LOR data including TOF information by:
using the TOF information to determine an occurrence probability that an annihilation event represented by an LOR is located within the projection mask; and
if the occurrence probability indicates that the annihilation event represented by the LOR is located within the projection mask, then using the LOR to trace image elements not having the out-of-boundary value.

27. The article of manufacture 4 of claim 20, wherein the computer readable program, when executed on the computer, further causes the computer to reconstruct the identified list mode events by:
using annihilation event TOF information to determine an occurrence probability that an event represented by an LOR is located within the projection mask; and
if the occurrence probability indicates that the annihilation event represented by the LOR is located within the projection mask, then using the LOR to trace image elements located in the region of interest.
